(19) United States
(12) Patent Application Publication
(10) Pub. No.: US 2023/0355361 A1
(43) Pub. Date: Nov. 9, 2023

(54) DENTURE IMAGING AND MODELING USING CT SCANNING DEVICES

(71) Applicant: VOYAGER DENTAL, INC., Edina, MN (US)

(72) Inventors: John MADDEN, Minneapolis, MN (US); Michael C. MARSHALL, Edina, MN (US); Stephen B. Siegfried Floe, Chanhassen, MN (US)

(21) Appl. No.: 18/314,210

(22) Filed: May 9, 2023

Related U.S. Application Data
(60) Provisional application No. 63/339,894, filed on May 9, 2022.

(51) Int. Cl. A61C 13/00; A61C 9/00

(52) U.S. Cl. CPC A61C 13/0004; A61C 9/0053

(57) ABSTRACT

A method for generating a digital denture model can include receiving scans of dentures generated by a CT scan. The scans can include a bite scan showing the positioning of an upper and lower denture in a patient bite orientation. The bite scan can be used to position upper and lower denture scans in the patient bite orientation and a denture model can be generated from the upper and lower scans in the patient bite orientation. Irregularities in the denture model, such as voids and metallic inclusions, can be corrected to provide a smooth model surface. Color images of the dentures can be correlated with the denture model to provide a colored denture model. During the CT scan, prompts can be generated for displaying quality analysis information during scanning, recommendations for adjustments to equipment or orientation of the dentures, and steps for performing the scan.

### INCORPORATION BY REFERENCE

This application claims priority to U.S. Provisional Application Ser. No. 63/339,894, filed on May 9, 2022, the disclosure of which is incorporated by reference in its entirety.

### TECHNICAL FIELD

This document generally describes technology related to use of computed tomography (“CT”) scanning devices to image and model dentures.

### BACKGROUND

A denture is a dental prosthesis that is made to replace missing teeth. Dentures are often supported by the surrounding soft and hard tissue of the oral cavity. For example, a denture may be designed to fit over and be supported by a patient's gum tissue. Dentures may include a denture base region that is formed from an acrylic material and colored to appear similar to gum tissue. Denture teeth formed from acrylic or other materials may be secured to the denture base.

### SUMMARY

This document generally describes technology for using CT scanners to efficiently generate high-quality digital models of dentures, which can be used for a variety of purposes, such as creating new and/or replacement dentures for patients. Conventional techniques for creating digital models of dentures have relied on intraoral scanners, which scan the surface of a patient's dentures to generate a digital model of the dentures. Intraoral scanners, however, can have a variety of drawbacks, including taking a longer period of time (e.g., 10 or more minutes) to create a sufficient digital model of the dentures. Additionally, while intraoral scanners can produce high resolution digital models of dentures that accurately model the surface of a patient's dentures, such high resolution digital models may be more detailed than necessary to create new and/or replacement dentures. Additionally, such high resolution digital models can be sufficiently large in size (e.g., size of data file, memory required to store and/or render the models) that performing various operations using the model, such as 3D proximity and collision detection (e.g., iterative closest point (“ICP”)) with other modeled objects, may not be possible in real time using conventional computing hardware. The disclosed technology provides solutions to these and/or other problems by using CT scanners, which are present in many dental offices, to more quickly scan a patient's dentures (e.g., scan in 1-2 minutes as opposed to the longer period of time for intraoral scanners) and to generate digital models of the dentures that is sufficiently detailed yet small enough in size that it can be used in real time for various tasks, such as creating new and/or replacement dentures. Additionally, the digital models of dentures generated using the disclosed technology can accurately model a full set of dentures, including both the upper and lower dentures as well as their relative alignment with regard to each other, which can be used to more accurately create new and/or replacement dentures that will provide an a good fit within a patient's mouth.

For example, the disclosed technology can perform CT scans of existing dentures, including dentures with scans of existing dentures and create denture models from the scans. The disclosed technology can enable efficient capture of the denture data from CT scanners and processing of the scans to form models for use in preparing new dentures. The technology includes mechanisms to aid in performing quality scans using CT and CBCT machines that are used to create denture models for well-fitting dentures. The technology also includes mechanisms for processing of the scan data to form and refine the denture models.

Models of dentures are used to create replacement dentures for patients who have lost or damaged their dentures, or who would like an additional set of dentures in the future. Dentures need to be properly fitted to the soft tissue in a patient's mouth for comfort and proper function. The physical appearance, including shape, color, and pattern of the dentures can also be important to the patient. Additionally, preparing a scan of existing dentures can be time consuming for a dentist or technician. Additionally, if a scan is incomplete or poor quality the replacement dentures created from the scans can be ill-fitting or non-functional. A patient may need to return to the dentist for more scans, requiring additional time in the dental office and additional time and work for dental staff and technicians. The disclosed technology, as described throughout this document, can provide solutions to these and/or other problems with existing denture scanning and modeling technology.

For example, in one implementation, a method of using a CT scanning device to obtain scanned denture data can include positioning an upper denture and lower denture together in a patient bite orientation, positioning the upper and lower denture in the patient bite orientation in a scanning area of the CT scanning device, performing a CT scan with the CT scanning device, confirming an identification of the CT scan as the patient bite orientation in a user interface coupled to the CT scanning device, and receiving, in the user interface, a quality indication associated with the performed CT scan.

Another method of generating a digital denture model includes providing, on a user interface associated with a CT scanning device, a prompt to position an upper denture and a lower denture together in a patient bite orientation in a scanning area of the CT scanning device, performing a CT scan of the upper denture and the lower denture together in the patient bite orientation using the CT scanning device, receiving a bite scan based on the CT scan of the upper denture and the lower denture together in the patient bite orientation, and performing a quality analysis of the bite scan and providing a result of the quality analysis on a display.

Another method of preparing a digital denture model includes receiving scan data includes bite scan comprising a scan of an upper denture positioned with a lower denture in a patient bite orientation, an upper denture scan, and a lower denture scan, forming a bite model, an upper denture model and a lower denture model from the received bite scan, upper denture scan, and lower denture scan, determining a relative position of the upper denture model and the lower denture model based on the bite scan, and generating a denture model based on the upper denture model, lower denture model, and determined relative position.

Another method of preparing a digital denture model includes receiving an upper denture scan and a lower denture scan, forming an upper denture model and a lower denture model from the received upper denture scan and the received lower denture scan, determining an initial bite position of the upper denture model and the lower denture model, and generating a denture model based on the upper denture model, the lower denture model and the initial bite position.

Another method of preparing a digital denture model, includes receiving a bite scan comprising a scan of an upper denture and lower denture positioned together in a patient bite orientation, forming a bite model from the received bite scan, separating the bite model into an upper denture model and a lower denture model, and generating a denture model based on the upper denture model, the lower denture model, and a positioning of the upper denture model and lower denture model based on the bite model orientation.

Certain implementations may provide one or more advantages. In a first example, using a CT scanner to produce scans of dentures or denture records improves efficiency over use of intraoral scanners (“IOS”) or desktop scanners. The CT scanner can obtain a complete scan of the dentures in one, two, or three scans using equipment already present in many dental offices. Using a CT scanner to create scans of dentures can be two to five times faster than using an IOS or desktop scanner.

In a second example, the use of a CT scanner can enable the standardization of the process of producing a scan for creating a denture model. The quality of scans produced by IOS and desktop scanners are more dependent on the technique of the technician creating the scan because the scans are at least partly manually obtained. An IOS scan is formed from multiple images taken while a technician moves the IOS around the dentures, and a desktop scan requires the technician to move or rotate the dentures within the scanner periodically to capture all angles. In contrast, after placing the dentures in the scanning area of the CT scanner, the CT scanner can perform a single scan for use in forming the denture model, though in some cases additional scans may be useful. Because CT scans require less manual manipulation of the dentures, there is less difference between the work of technicians.

In a third example, the ability to provide simple prompts for use of the CT scanner and perform a scan by pushing a button enables automation of the procedure so that less training is required of technicians to perform the scan. The CT scanner interface can provide prompts to facilitate the scan process, and can also assess the quality of the scans and make recommendations to improve the scan quality, for example, by re-positioning the dentures, adjusting parameters of the CT scanner like the field of view, and re-scanning the dentures. The interface can also prompt the user to identify the scans and specify consistent file names for efficient identification of the denture scans. The dentures scans from the CT scanner can then be converted to an accessible file type and used to prepare a denture model. The CT scanner produces good quality denture scans that can create denture models for fabrication of well-fitting and comfortable dentures with high reliability.

In a fourth example, the use of a CT scanner increases processing efficiency as compared to IOS or desktop scanners. The CT scanner produces a scan that may be relatively rough compared to IOS or desktop scanners (which can be more precise), but the CT scanner produces a scan with sufficient details for most denture models and requires less memory for storing the scans and less processing power for capturing and manipulating the scans as compared to IOS or desktop scanners. IOS and desktop scanners can capture multiple (sometimes hundreds) of images which are then stitched together into a composite scan. In contrast, the CT scanner can produce a single scan.

Furthermore, as a fifth example, the CT imaging techniques described herein provide for faster processing and efficiency in generating accurate digital models for patient dentures. Although CT scanners may provide lower quality scans (e.g., parts of a scan may be missing, such as overlapping surfaces being blocked by some scan data like teeth), high quality scans are not required to generate accurate digital models for dentures and other dental appliances. In other words, accurate digital models for dentures and dental appliances can be generated using data that is low quality and/or incomplete. Similarly, because the disclosed techniques are faster and more efficient, users with lower skillsets than doctors, dentists, and other technicians may perform these techniques to generate accurate digital models for dentures and other dental appliances. As a result, dentures and other dental appliances can be more efficiently and quickly designed, manufactured, and installed for patients, without requiring the patients to return multiple times to a dental office to have their dentures or dental appliances fixed/adjusted.

Additional advantages will be apparent to the person of skill in the art based on the figures, description, and claims herein.

### DESCRIPTION OF THE DRAWINGS

FIG. 1A is a diagram of an example process for preparing replacement dentures using CT scans.

FIG. 1B is an exemplary view of a CT scanner used to perform a scan of existing dentures.

FIG. 1C is an exemplary view of dentures positioned in a scanning area of a CT scanner.

FIGS. 2A, 2B, and 2C are views of denture models obtained by CT scanning.

FIG. 3 is a flowchart of an example process for obtaining CT scan denture data with a CT scanner.

FIG. 4 is a flowchart of an example process for obtaining CT scan denture data with a CT scanner.

FIG. 5 is a flowchart of an example process for using the CT scan denture data to generate a model of the dentures.

FIG. 6 is a flowchart of an example process for obtaining and using CT scan denture data for generation of denture models.

FIG. 7 is a flowchart of an example process for obtaining and using CT scan denture data for generation of denture models using bite registration material.

FIG. 8 is a flowchart of an example process for performing denture scans following prompts in a user interface.

FIG. 9 is a flowchart of an example process for using CT scan denture data and color images of the scanned dentures to generate a denture model.

FIG. 10 shows a comparison of flowcharts illustrating a conventional denture fabrication workflow and an example workflow for preparation of dentures using CT scanning.

Like reference symbols in various drawings indicate like elements.

### DETAILED DESCRIPTION

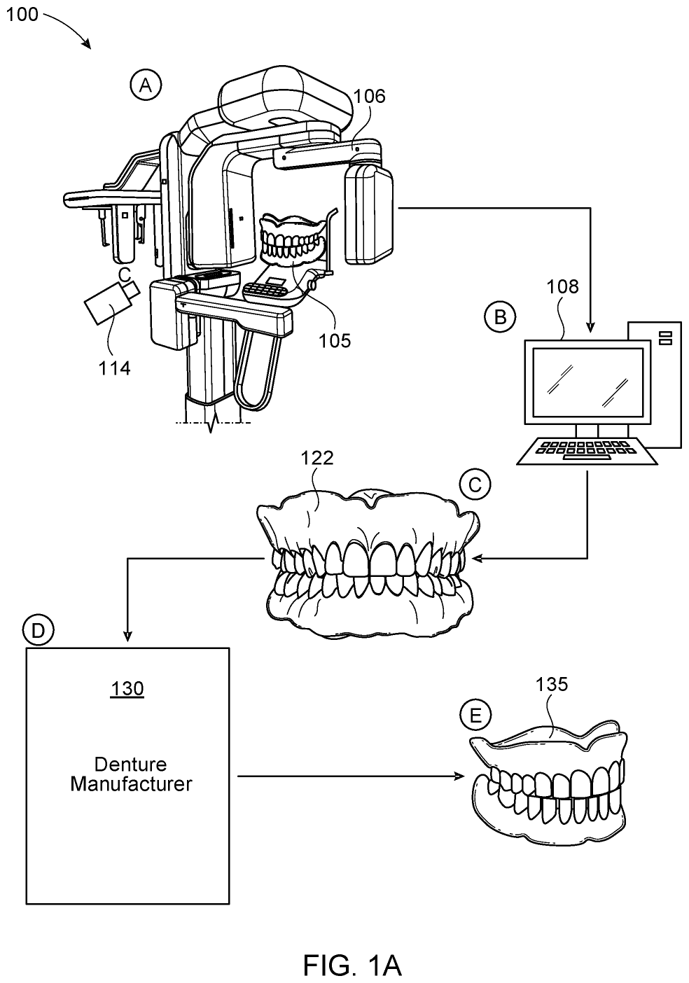

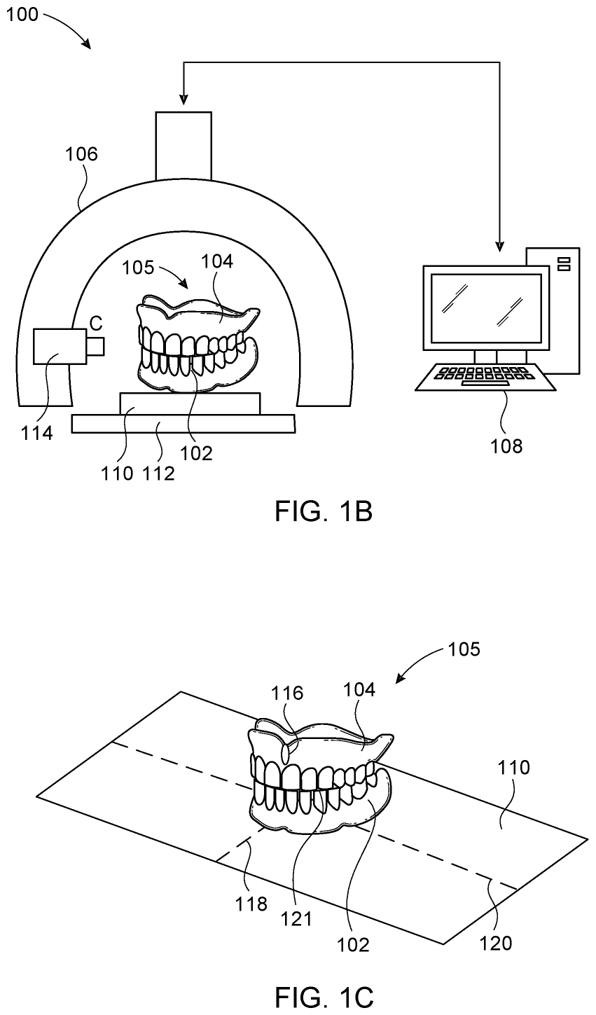

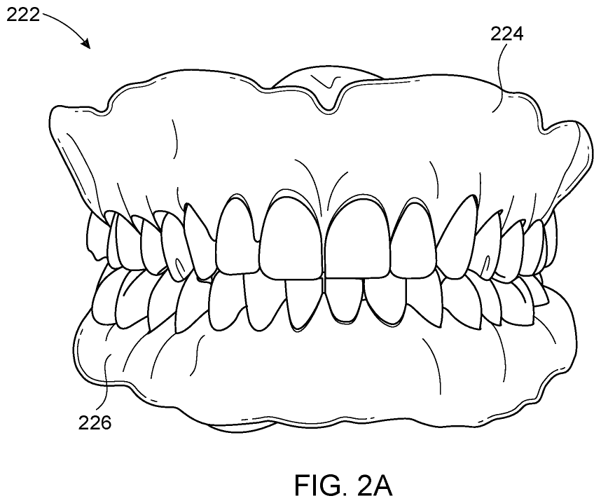

FIGS. 1A-C show views of an exemplary process for obtaining CT scans of dentures or denture records/references and using the scans to create accurate denture models. Referring to FIG. 1A, the process for preparing replacement dentures using CT scan data is illustrated. At step A, a CT scanner 106 performs scans of dentures 105 (or denture reference or records). In some implementations, a color camera 114 captures a color image of the dentures 105. In some implementations, the color camera 114 is integrated with the CT scanner 106, and the color camera 114 captures the color image simultaneously with the CT scanner 106 performing the scan.

At step B, the data from the CT scanner 106 and the color camera 114 is transmitted to a computer 108 or other data processing device. The computer 108 can include programs stored in a memory which are executable to analyze the scanned data from the CT scanner 106 and to process the scanned data to produce a denture model 122 at step C. The computer 108 can also provide a quality analysis of the scanned data and report the results of the quality analysis to a technician at the CT scanner to prompt the technician to alter the settings on the CT scanner, reposition the dentures, or perform a new scan.

The computer 108 can detect in the scanned data, irregularities such as voids or metallic inclusions, and can remove or correct the irregularities to produce a data model having a smooth surface. For example, some scans may have a textured surface or holes in the scan, which can be automatically fixed prior to generating a model from the scanned data. The computer 108 may perform a surface assessment and a hole assessment, and can recommend in a user interface that an additional scan should be performed where the surface assessment and hole assessment indicate poor quality of the scan. The computer 108 can process the data to convert it from the native file type of the CT scanner (for example, a Digital Imaging and Communications in Medicine (DICOM) file) to a data model file type such as a stereolithography (STL) file type. In some implementations, the DICOM file or other native file of the CT scanner is converted to a polygon file format (PLY), a standard 3D image file format (OBJ), an additive manufacturing file format (AMF), a 3D manufacturing file format (3MF), or any other suitable file type.

Once the computer 108 has processed the scanned data to produce a denture model 122, the denture model 122 can be sent to a denture manufacturer 130 at step D. The denture manufacturer produces a set of replacement dentures 135 by milling or printing techniques.

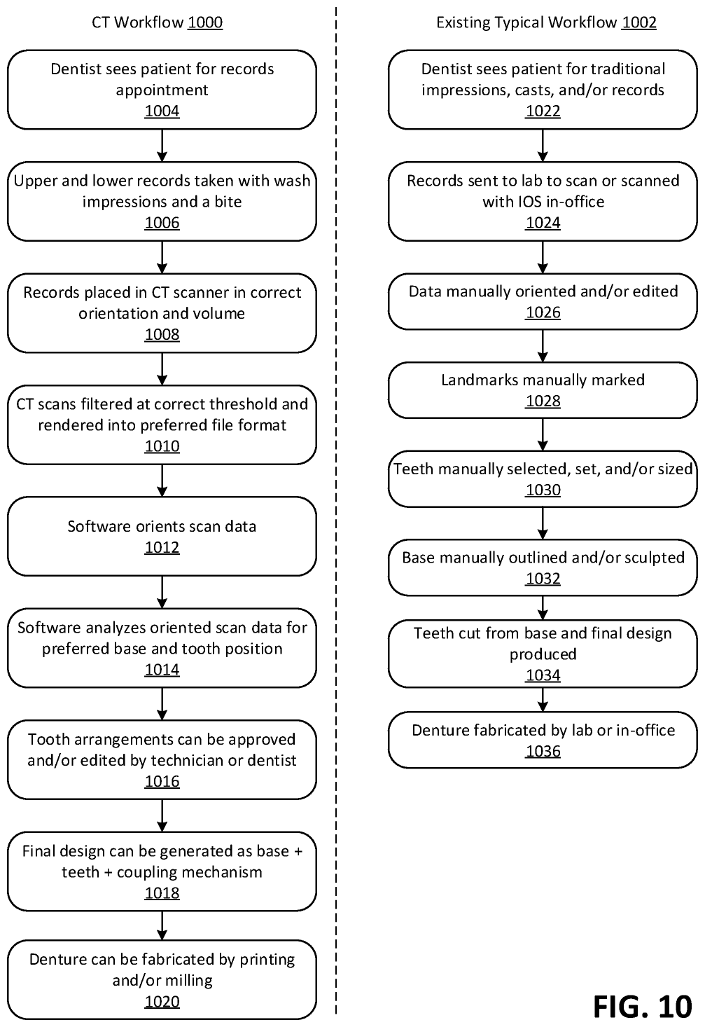

FIG. 10 describes the exemplary process of FIG. 1A in more detail. FIG. 10 shows a flowchart illustrating example CT workflow 1000 for preparation of dentures using CT scanning in comparison to an existing typical workflow 1002.

Referring to the CT workflow 1000, the process 1000 begins with the dentist collecting records in the office with the patient (1004). There are many possible structures that can be used as starting points in the process, including reference or replacement dentures, “mono-block” try-ins (biofunctional try-ins), wax rims, or hybrid wax rims try-ins. Any of these structures can be used for the collection of scan data in the CT scanning process. References herein to existing dentures or dentures can mean any of the structures that form the starting point for the generation of new or replacement dentures.

In one example, the process 1000 begins with the dentist meeting with the patient to collect denture records. In some cases, using the reference dentures, the dentist will perform a wash impression using polyvinyl siloxane (PVS) or vinyl polysiloxane (VPS) wash impression materials to capture an accurate and stable record of the tissues. A wash impression is performed in both arches of the reference denture (if both arches are to be fabricated). The wash impression is made of a similarly dense material to the denture, for example, a material that is radio opaque at a desired threshold.

Once the wash is performed on both arches, a bite record can be taken to record the relative position of the upper arch to the lower arch (1006). This bite record is most commonly made from VPS (or PVS) material but could be made from radio translucent material. The bite record is produced to record the ideal position of the upper denture in relation to the lower denture in the patient's mouth. This means the bite record may account for desired changes in the vertical dimension or jaw position and worn occlusion, and can correct deficiencies in the reference denture.

After the bite record is taken, material can be trimmed to eliminate any overhang on the teeth (or other landmarks) that would obstruct the scan. Obstruction of the scan could lead to poor results, inaccuracies, and errors later in the process making automation difficult. Once the bite record material is trimmed, the upper arch, bite record, and lower arch are assembled together in the correct orientation and purposefully placed facing forward on an appliance scanning jig in the CT scanner. A fiduciary may also be attached in precise position on the assembled upper arch, bite record, and lower arch, and used to determine a precise position of the assembly within a scanning area of the CT scanner.

In some implementations, the records/reference dentures are placed on low density foam to form a layer between the appliance and the records so that supporting structures of the jig do not obstruct the CT scan of the reference denture. For stability, it may also be beneficial to place the upper and lower reference dentures upside-down on the jig (but still facing forward).

After the records/reference dentures are securely positioned in the scanning area of the CT scanner (1008), so that they are stable and positioned in the correct direction and predictable orientation, the ideal volume size and threshold can be selected in the CT scanner interface. In many cases, the volume size and threshold required for scanning the reference dentures may be larger than a normal appliance scan volume. A slightly larger volume size will allow for the upper and lower arch to be scanned at the same time. In some implementations, a specific threshold of 800-1,100 is selected in the CT scanner to scan both the upper and lower arch at the same time. In some implementations, an optimization algorithm is applied to find the correct surface rendering for both the reference dentures and the VPS wash impressions.

After assigning the correct volume the CT scan can be performed. The scan is then modified by application of a threshold to the scan (1010), so that the ideal surface is rendered into a STL file. One or more other file types may be used, such as PLY. The STL type can advantageously allow for smaller data transfer and/or faster manipulation. Thus, this file type is relatively small when compared to normal DICOM CT data and therefore can easily be communicated and manipulated efficiently.

Once the CT scan is performed, software or other computing techniques described herein can orient the scan data (1012). The software can also analyze the oriented scan data for determining a preferred base and/or tooth position for the patient (1014). In some implementations, determining the preferred tooth position(s) for the patient can include leveling and/or aligning teeth to arch forms and/or occlusal planes, snapping teeth to a particular fit, individually moving the teeth along one or more of the upper and lower arches, and/or using color maps to visually display movement of teeth changes, which are described further in U.S. Provisional Patent Application No. 63/348,217, entitled “Auto-Denture Design System for Denture Replacement” filed on Jun. 2, 2022 (the '217 application), U.S. patent application Ser. No. 17/876,090, entitled “Tools and Automation for Tooth Setup” filed on Jul. 28, 2022 (the '090 application), PCT Patent Application International App. No. PCT/US22/16362, entitled “Digital Denture Design and Replacement” filed on Feb. 14, 2022 (the '362 application), and U.S. patent application Ser. No. 18/174,361, entitled “Color Digital Denture Design and Replacement” filed on Feb. 24, 2023 (the '361 application), all of which are incorporated herein by reference in their entireties. In some implementations, the base can be designed using techniques such as automatically generating a base based upon user selection of an option to do so and/or automatically suggesting one or more base designs based on the patient's scan data, as described further in reference to “Auto-Denture Design System for Denture Replacement” (the '217 application), “Tools and Automation for Tooth Setup” (the '090 application), and “Digital Denture Design and Replacement” (the '362 application), all of which are incorporated herein by reference.

The preferred positioning of the base and/or teeth can then be presented to the dentist, technician or other relevant user in a user interface as described herein. The user interfaces described herein can include any of the graphical user interfaces (GUIs) described in reference to “Auto-Denture Design System for Denture Replacement” (the '217 application), “Tools and Automation for Tooth Setup” (the '090 application), “Digital Denture Design and Replacement” (the '362 application), and “Color Digital Denture Design and Replacement” (the '361 application), which are incorporated herein by reference in their entireties. Such tooth and/or base arrangements can be approved and/or edited by the dentist in the user interface (1016). For example, the dentist can provide user input using user-selectable features and/or controls to modify arrangement of one or more teeth and/or the base for the particular patient. Then, a final design can be generated by the software as a base, teeth, and/or coupling mechanism (1018). The final design can be transmitted to a device and/or machine for fabricating dentures according to the final design for the patient (1020). For example, a rapid fabrication machine can be used to manufacture the dentures for the patient according to the final design. The dentures can be fabricated by executing printing and/or milling techniques according to execution instructions associated with the final design.

Referring now to the existing typical workflow 1002, this process 1002 can begin with the dentist seeing a patient for traditional impressions, casts and records (1022). The records are then sent to a lab to scan or are scanned with an intraoral scanner in the dental office (1024). The data from the scan is then manually oriented and edited (1026), landmarks in the scan are manually marked (1028), teeth are manually selected, set and/or sized (1030), and the base can be manually outlined and/or sculpted (1032). The teeth can then be cut from the base and the final design can be produced (1034), after which the denture may be fabricated by an external lab or in the dental office (1036). Compared to the conventional workflow of the process 1002, the workflow of the process 1000 can produce more standardized and precise dentures because of automation of many aspects that are manually inputted according to the traditional or convention process 1002. The dentures fabricated using the models generated by the workflow in the process 1000 are more likely to fit and function as desired for the particular patient, and can be prepared more quickly and efficiently, without the need for the patient to make multiple visits to the dental office. Furthermore, because the workflow of the process 1000 leverages CT scans and other lower quality-type scan data, the process 1000 can provide for more efficiently and quickly generating models of dentures in comparison to the conventional process 1002, in which human actors are required to process and manually manipulate data representing the patient's mouth. The workflow of the process 1000 is described further below.

FIG. 1B is an exemplary view 100 of dentures 105 positioned in a CT scanner 106. The CT scanner 106 includes a scanning area 112 and, optionally, a color camera 114. The CT scanner 106 is in communication with a computer 108. In some implementations, the CT scanner 106 is a cone beam CT scanner.

The dentures 105 include an upper denture 104 and a lower denture 102. As described above, the dentures 105 can be an existing pair of dentures, reference or replacement dentures, “mono-block” try-ins (biofunctional try-ins), wax rims, or hybrid wax rims try-ins. The dentures 105 are positioned on a structure 110 within the scanning area 112 of the CT scanner. In some implementations, the structure 110 is a foam structure. In some implementations, the structure 110 is a denture rig designed to support dentures 105 in the scanning area 112. In some implementations, the structure 110 is formed from a material having a known density. In some implementations, the structure 110 is formed from a material that is radio-opaque or radio-translucent.

As illustrated in FIG. 1C, the structure 110 can include fiducials or markings to aid in aligning and positioning the dentures 105 for scanning. FIG. 1C is an exemplary view of dentures 105 positioned on a structure 110 for scanning by a CT scanner 106. The structure 110 includes one or more markings 118 and 120 on a surface of the structure 110 to help a technician position the dentures 105 in the scanning area 115. Marking 118 is a line on the structure 110 that identifies an appropriate positioning for a midline of the dentures 105. Marking 120 is a line on the structure that intersects with marking 118 to indicate an appropriate position on which to center the dentures 105 in the scanning area. The appropriate positioning of the dentures 105 can ensure that both the upper denture 104 and the lower denture 102 are captured by a scan. Alternatively, instead of markings on the structure 110, the structure 110 could include a light guide, a laser stripe, a scribe line, a piano wire, sticker, or any other suitable indication of proper positioning on the structure 110. Additionally or alternatively, the computer 108 can identify a midline of the dentures 105 based on an identification of landmarks such as teeth, cusp tips, or other identifiable portions of the dentures. The landmarks can be identified using any of the techniques described in “Auto-Denture Design System for Denture Replacement” (the '217 application), “Tools and Automation for Tooth Setup” (the '090 application), “Digital Denture Design and Replacement” (the '362 application), and “Color Digital Denture Design and Replacement” (the '361 application), which are incorporated herein by reference, in their entireties.

Appropriately positioning the dentures 105 on the structure 110 and in the scanning area 115 can also aid in processing the orientation of the scanned data relative to a known location in the scanning area 115. The markings 118 and 120 can aid in the positioning of the dentures 105, or can be included in the scanned data to orient the dentures 105 during processing. Alternatively or additionally, a fiducial sticker 116 can be added to the dentures 105 prior to scanning. The fiducial sticker 116 can indicate a midline on the dentures for use in processing. The fiducial sticker 116 can also aid determining a size of one or more dimensions of the dentures during processing. The fiducial sticker 116 can be formed from a material that a radio-opaque or that has a known density that differs from a density of the materials of the dentures 105. For example, the fiducial sticker 116 can have a high density material on or in the sticker that can be easily identified in the scan.

To provide a CT scan with the upper denture 104 and lower denture 102 oriented with respect to one another with the bite orientation that they have when worn by a patient, a bite registration material 121 can be used. To use a bite registration material 121, a dentist uses a material to take a bite record from a patient wearing the dentures. The bite registration material 121 can then be inserted between the upper denture 104 and lower denture 102 to space the dentures apart or to hold them in the orientation that they will have in the patient's mouth. Scanning the dentures in this orientation can help to generate a denture model for replacement dentures that are well-fitting and comfortable for the patient. While the dentures 105 are shown with the upper denture 104 oriented above the lower denture 102 and the lower denture 102 in contact with the structure 110, in some implementations, it may be advantageous to orient the dentures 105 with the upper denture 104 in contact with the structure 110 for stability.

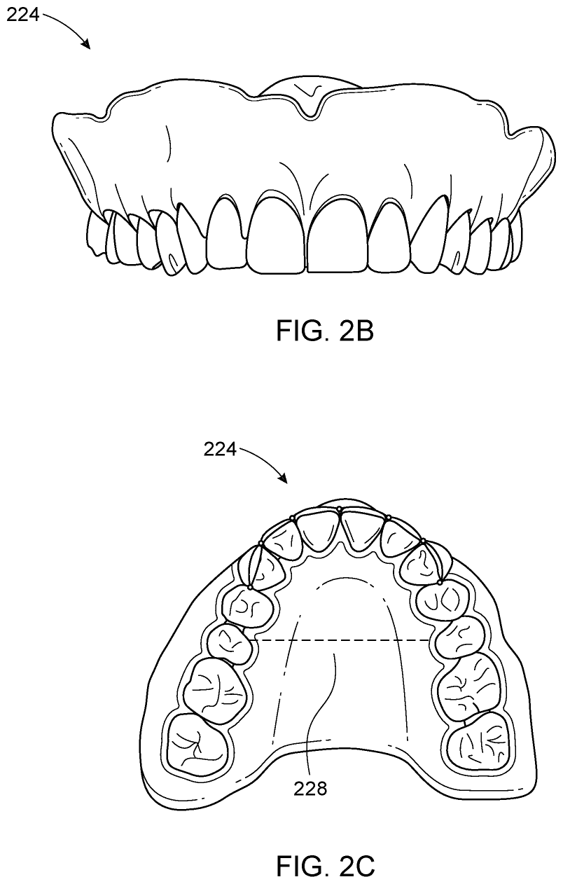

FIGS. 2A-C are views of CT scans of dentures. The CT scans include in FIG. 2A a bite scan 222 with an upper denture 224 and a lower denture 226. FIG. 2B shows a view of an upper denture 224 alone, and FIG. 2C shows an alternate view of the upper denture 224 showing the arch of the teeth, and a width of the arch 228. In some implementations, the bite scan 222 can include a bite registration material, which can be removed in the processing by identifying the density of the bite registration material and adjusting thresholds to remove the particular density of the bite registration material from the scan so that the bite registration material will not appear in the model.

In some implementations, only a bite scan 222 is captured by CT scanning, and the upper denture 224 and lower denture 226 are produced by separating the bite scan 222 into the two components. In some implementations, additional CT scans of the upper denture 224 and lower denture 226 are also performed to improve the detail captured for the upper denture 224 and lower denture 226. In some implementations, the computer program can identify which of the bite scan 222, upper denture 224 and lower denture 226 a particular scan represents based on the relative sizes of the scans, including a width 228 of the arch.

Modern procedures for denture fabrication include a process of acquisition of denture information by scanning the denture, followed by computer-aided design and fabrication through printing, milling, or a combination of the two. Efficient processes that save time benefit clinical and institutional outcomes. Table 1 shows a comparative illustration of denture processes.

TABLE 1
comparative illustration of denture processes.

| | IOS | DESKTOP | CT METHOD |
|---|---|---|---|
| TIME | 10-15 minutes | 10-15 minutes | 2-5 minutes |
| PERSONNEL | Dentist | Technician | Assistant or Staff |
| FILE TYPE | STL, PLY | STL | DICOM |

Use of an intraoral scanner (labeled “IOS” in Table 1) in the clinic or desktop scanner (labeled “Desktop” in Table 1) in the laboratory each require about 10-15 minutes of dentist or technician time. In contrast, using a CT scanner (labeled “CT method” in Table 1) as described in this document for the same acquisition of denture records offers a significant time savings and can be accomplished in a few minutes. When the time savings achieved by use of CT scanner are applied across an entire institution or organization that is heavily involved in the clinical production of dentures, the more efficient procedure is even more advantageous. For example, a large dental service organization may produce tens of thousands of dentures a year and therefore save thousands of hours of clinician and staff time per year by adopting a method using CT scans.

As shown in Table 1, not only does the CT method take the least amount of time, but also requires the least costly staffing to complete the procedure. Furthermore, it results in less chair time to complete the appointment which in turn allows for more daily appointments and bookings.

In addition to these timesaving benefits, using a CT scanner for the acquisition of denture records allows for standardization and automation in the design phase. If the dentures being scanned are inserted into the scanner in a specific fashion, the midline of the denture and orientation of the scans can be automated instead of having to be done manually by a technician. To better facilitate this automation, a fiduciary marker with unique shape may be placed on the denture during the scan.

After the orientation of the scan is determined and the position of the midline is established, the automation of much of the design is possible. Software can determine the appropriate tissue facing side of the denture and propose an initial tooth-arch position. The proposed tooth arrangement can then be edited by a technician or dentist and the final design milled or printed locally in the office.

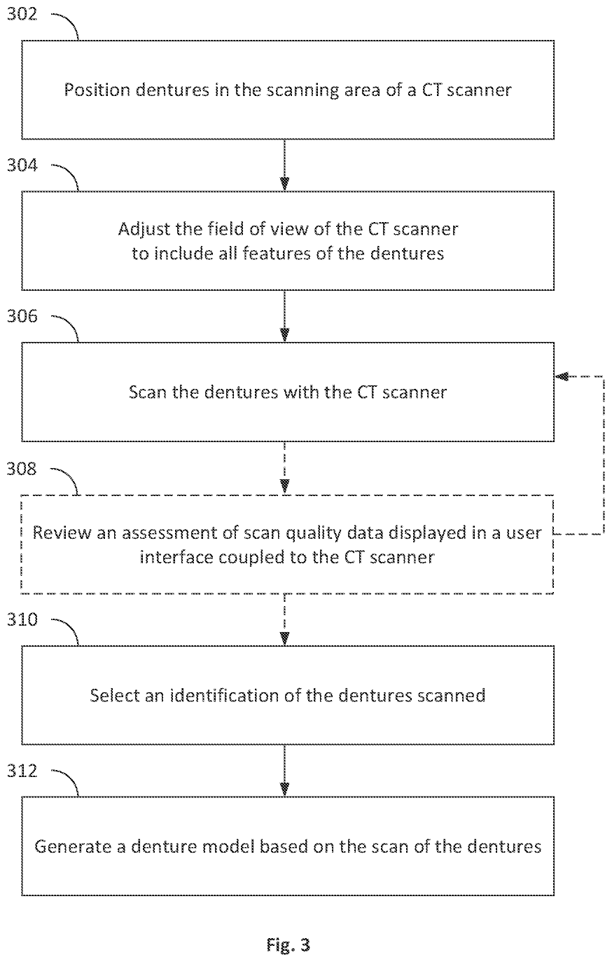

FIG. 3 is a flowchart of an example process for obtaining CT scan denture data with a CT scanner. At step 302, dentures are positioned in the scanning area of a CT scanner. At step 304, the field of view of the CT scanner is adjusted to include all features of the dentures. Additional parameters of the CT scanner may also be adjusted, for example, the threshold may be set for a particular density of interest. At step 306, the dentures are scanned with the CT scanner. Optionally, at step 308, an assessment of scan quality data is displayed in a user interface coupled to the CT scanner, and the user can review the assessment and perform additional scans if indicated by the assessment (i.e., return to step 306). In some implementations, the scan quality data assessment includes a recommendation to re-scan the dentures, re-adjust the field of view of the CT scanner, and/or reposition the dentures in the scanning area of the scanner. In some implementations, the scan quality data assessment includes display of a view of the CT scan for the user to review.

At step 310, an identification of the dentures scanned is selected. In some implementations, the identification is suggested in the user interface, for example by identifying a scan as likely a scan of an upper denture alone or a bite scan. In such cases, the user of CT scanner may confirm in the user interface the identification of the scan. In some implementations, the user selects a button or manually enters a filename including an identification of the scan. At step 312, a denture model is generated based on the scan of the dentures.

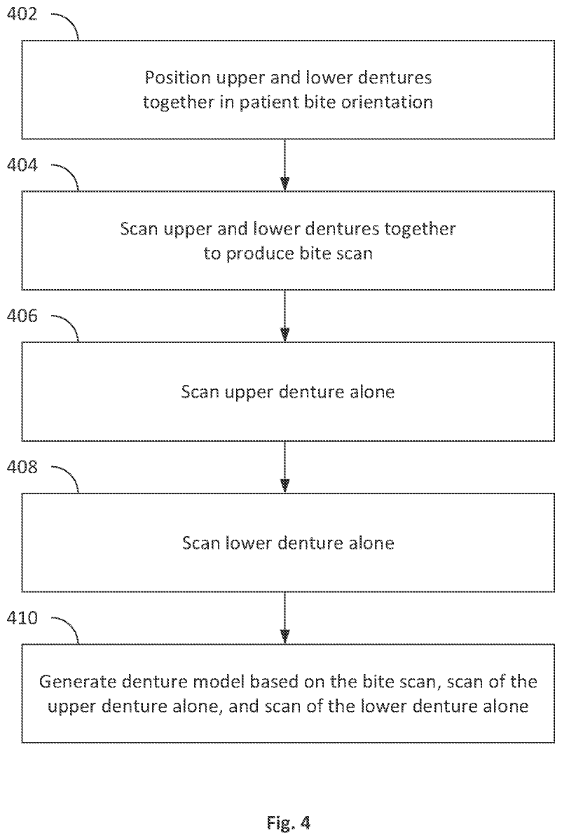

One or more scans of the dentures can be used to produce a denture model. For example, FIG. 4 describes a process of using three scans of the upper denture, lower denture, and both dentures in a patient bite orientation to generate a denture model. Referring to FIG. 4, at step 402, the upper and lower dentures are positioned together in a patient bite orientation. At step 404, the upper and lower dentures are scanned together to produce a bite scan. At step 406, the upper denture alone is scanned. At step 408, the lower denture alone is scanned. In some implementations, the field of view required for the CT scan of the bite scan is larger than the field of view required for the scans of the upper and lower dentures alone.

At step 410, a denture model is generated based on the bite scan, the scan of the upper denture alone, and the scan of the lower denture alone. For example, in some implementations, an iterative closes points (ICP) algorithm is used to find the best fit of the upper and lower denture scans to the bite scan. The bite scan provides the relationship between the upper and lower dentures, while the details of each denture is determined from the scan of the upper and lower dentures alone.

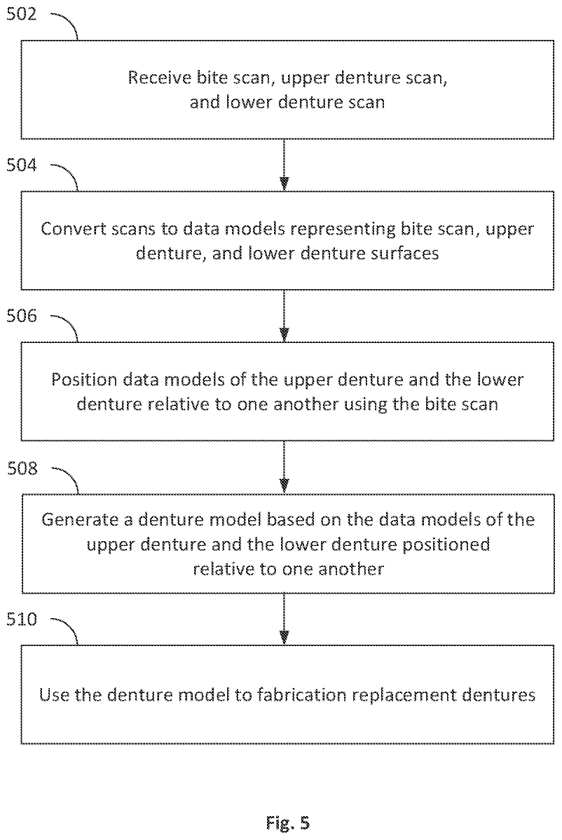

FIG. 5 describes how the scan data can be used to generate the denture model. At step 502, the bite scan, upper denture scan, and lower denture scan are received. At step 504, the scans are converted to data models representing the bite scan, upper denture and lower denture surfaces. At step 506, the data models of the upper denture and the lower denture are positioned relative to one another using the bite scan. At step 508, a denture model is generated based on the data models of the upper denture and the lower denture positioned relative to one another. At step 510, the denture model is used to generate a replacement denture. Alternatively, the denture model can be used for a variety of other purposes, for example to visualize one or more changes to the dentures, to keep in case another denture is needed later, or any other suitable purpose.

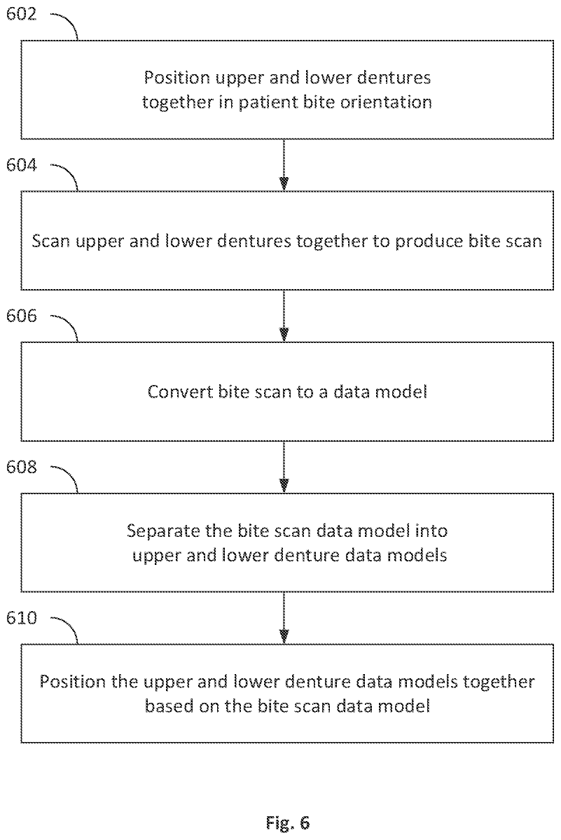

In some cases, only one scan may be needed to produce a denture model. For example, FIG. 6 is a flowchart of an example process for obtaining and using just a CT scan of the upper and lower dentures in a bite orientation to generate a denture model. At step 602, the upper and lower dentures are positioned together in a patient bite orientation. At step 604, the upper and lower dentures are scanned together to produce a bite scan. At step 606, the bite scan is converted to a data model. At step 608, the bite scan data model is separated into upper and lower denture data models. At step 610, the upper and lower denture data models are positioned together based on the bite scan data model.

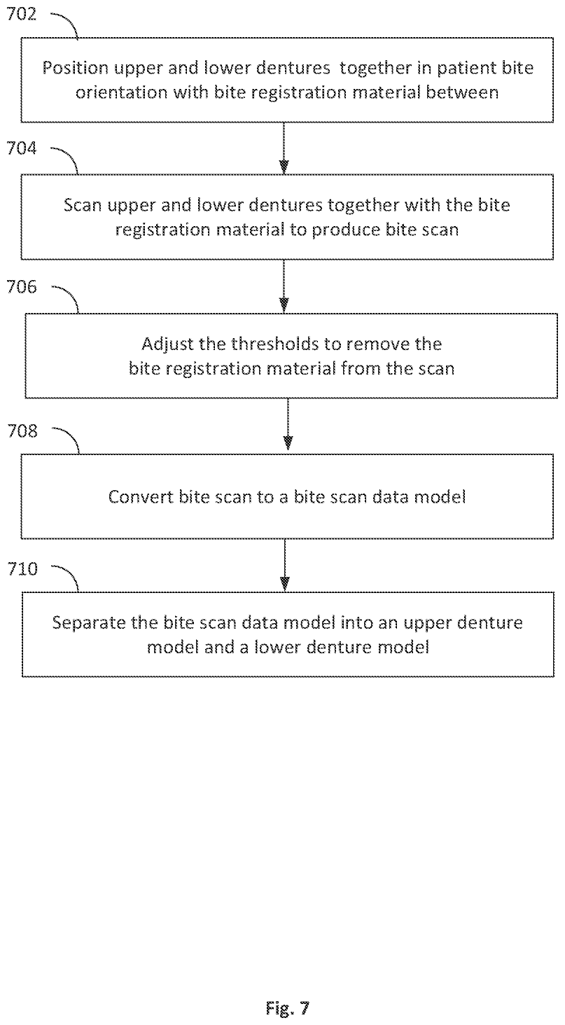

In some cases, the dentist hand articulates the upper and lower dentures into a bit configuration. In other cases, the patient's bite can be more easily matched or approximated, and the bite scan can be more easily separated into upper and lower denture models with the use of a bite registration material. A bite registration material can also allow a user to more accurately position the dentures as they would be positioned in the patient's mouth, providing a denture model that can be used to fabricate a comfortable denture that fits similarly in the patient's mouth as the original denture. FIG. 7 is a flowchart describing a process for obtaining and using CT scan denture data for generation of denture models using bite registration material. At step 702, the upper and lower dentures are positioned together in a patient bite orientation with a bite registration material between the upper and lower dentures. At step 704, the upper and lower dentures are scanned together with the bite registration material to produce a bite scan. At step 706, the thresholds are adjusted to remove the bite registration materials from the scan. Alternatively, if the bite registration material is radio-translucent, this step may not be necessary, as the bite registration material will not show up in the scan. At step 708, the bite scan is converted to a bite scan model. At step 710, the bite scan model is separated into an upper denture model and a lower denture model. In some implementations, a first bite scan is performed with the CT scanner with the upper and lower dentures hand articulated into a position to replicate the patient's bite, and a second bite scan uses a bite registration material to separate the upper and lower dentures from one another for ease of separation of the scans into separate components.

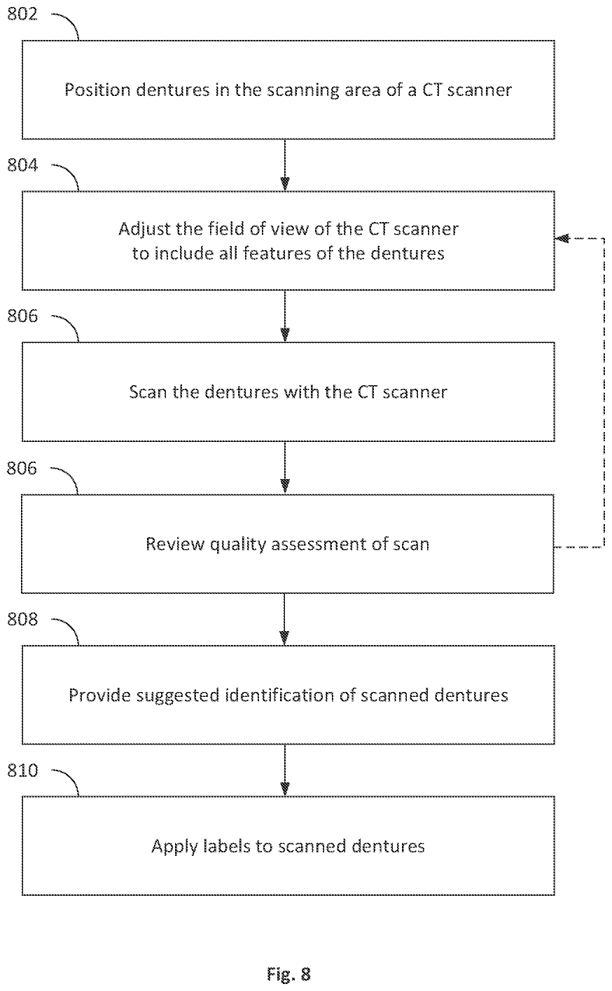

FIG. 8 is a flowchart of an example process for performing denture scans following prompts in a user interface. At step 802, the dentures are positioned in the scanning area of a CT scanner. In some implementations, a user interface can include one or more prompts to facilitate positioning the dentures in an appropriate position and orientation in the scanning area. For example, the prompts can include describing which of the upper and lower dentures should be positioned in the scanning area first, recommending an orientation of the dentures (e.g., facing forward, teeth side down), or recommending a positon of the dentures (e.g., centered on a structure, on a marking on the structure). At step 804, the field of view of the CT scanner is adjusted to include all features of the dentures. In some implementations, the user interface includes a prompt to adjust the field of view. In some implementations, the user interface prompt includes a recommendation to use a pre-set field of view, or suggests a range for the field of view to be used.

At step 806, the dentures are scanned with the CT scanner. At step 808, a quality assessment of the scan is presented on the user interface for review. The quality assessment can take a variety of forms, including one or more of a color or numeric representation of the quality, a display of the scan data to user, and a recommendation to make an adjustment to the CT scanner or dentures or to perform another scan. In response to the recommendations presented on the user interface, or based on the user review of the assessment, the user can choose to re-adjust the field of view and re-scan the dentures (return to step 804), or to re-position the dentures or perform a re-scan of the dentures without adjusting the field of view.

At step 808, a suggested identification of the dentures is provided in the user interface. For example, the user interface can provide options for a user to select between to identify whether a scan is of the upper denture, the lower denture, or both. Alternatively, the user interface can provide a particular suggested identification of the dentures based on an assessment of the scan data to determine whether the scan is likely of the upper denture, the lower denture, or both. The user can respond by confirming the suggested identification or choosing another identification. At step 810, labels are applied to the scanned dentures, including naming conventions. In some implementations, a user manually inputs one or more labels for the dentures in a user interface. In some implementations, the labels are automatically created and applied and the user confirms the labels are correct. In some implementations, the identification is automatically included in a file naming convention of the scan.

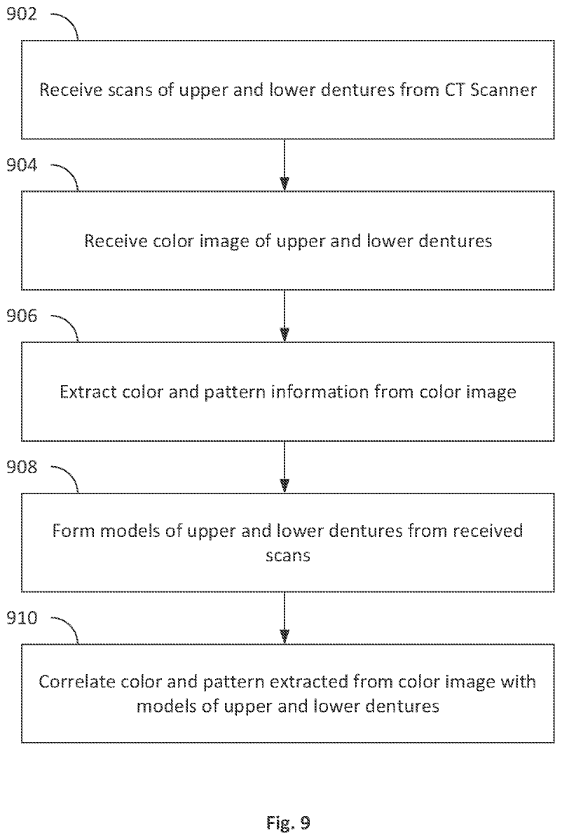

In some cases, color information can be simultaneously captured at the same time as the CT scan and the color information can be used to provide a color denture model for fabrication of replacement dentures. FIG. 9 is a flowchart of an example process for using CT scan denture data and color images of the scanned dentures to generate a denture model. At step 902, scans of the upper and lower dentures are received from the CT scanner. At step 904, the color image of the upper and lower dentures is received. At step 906, the color and pattern information is extracted from the color image. At step 908, models of the upper and lower dentures are formed from the received scans. At step 910, the color and pattern extracted from the color image is correlated with the models of the upper and lower dentures.

The association or mapping of the color image information to the denture model can be particularly useful for use by fabrication facilities using 3D printers capable of printing in multiple colors and densities. In some implementations, the color image is captured simultaneously with the CT scan. In some implementations, the color image is captured by a camera integrated with the CT scanner. In some implementations, the CT scanner also provides illumination of the dentures during the scan to enable an accurate color image to be captured. Additional details of the process are described in U.S. patent application “Color Digital Denture Design and Replacement” (the '361 application), “Auto-Denture Design System for Denture Replacement” (the '217 application), “Digital Denture Design and Replacement” (the '362 application), and U.S. Provisional Patent Application Ser. No. 63/313,723 filed on Feb. 24, 2022 and titled “Color Digital Denture Design and Replacement” (the '723 application), the contents of which are incorporated herein by reference in their entireties.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

The present disclosure is not to be limited by what has been particularly shown and described. One or more illustrative examples of the techniques described herein can include the following.

Example 1 can include a method of obtaining scanned data for a digital denture model, the method comprising: positioning an upper denture and a lower denture together in a patient bite orientation; positioning the upper denture and the lower denture in the patient bite orientation in a scanning area of a CT scanning device; obtaining, using the CT scanning device, a bite scan of the upper denture and the lower denture together in the patient bit orientation; positioning the upper denture in the scanning area of the CT scanning device; obtaining, using the CT scanning device, an upper denture scan; positioning the lower denture in the scanning area of the CT scanning device; obtaining, using the CT scanning device, a lower denture scan; and transmitting scan data comprising the bite scan, upper denture scan, and lower denture scan to a design software.

Example 2 can include the method of example 1, further comprising: placing a bite registration material between the upper denture and the lower denture prior to positioning the upper denture and the lower denture together in the patient bite orientation.

Example 3 can include the method of any one of examples 1-2, wherein the bite registration material is a radio-opaque material.

Example 4 can include the method of any one of examples 1-3, wherein the bite registration material is a radio-translucent material.

Example 5 can include the method of any of examples 1-4, wherein the bite registration material has a known density different than a density of a material forming the upper denture and the lower denture.

Example 6 can include the method of any of examples 1-5, further comprising: obtaining a color image of at least one of the upper denture and the lower denture together in a patient bite orientation, the upper denture, and the lower denture; and transmitting the color image to the design software.

Example 7 can include the method of any one of examples 1-6, wherein the color image is obtained by a camera coupled to the CT scanning device.

Example 8 can include the method of any one of examples 1-7, wherein the color image is obtained simultaneously with one or more of the bite scan, upper denture scan, and lower denture scan.

Example 9 can include the method of any of any one of examples 1-8, wherein the CT scanning device is a cone beam CT scanning device.

Example 10 can include the method of any of examples 1-9, further comprising: adjusting a field of view of the CT scanning device to include all features of the upper denture and the lower denture together in a patient bite orientation prior to obtaining the bite scan.

Example 11 can include the method of any of examples 1-10, further comprising: presenting, on a display, at least one of the bite scan, upper denture scan, and lower denture scan prior to transmitting the scan data to the design software.

Example 12 can include the method of any of examples 1-11, further comprising: presenting, on the display, an indication of scan quality with the at least one of the bite scan, upper denture scan, and lower denture scan.

Example 13 can include the method of any of examples 1-12, further comprising: prompting a user to identify each scan as one of the bite scan, the upper denture scan, and the lower denture scan.

Example 14 can include the method of any of examples 1-13, further comprising: prompting a user to position one or more of the upper denture and the lower denture relative to an indicator in the scanning area of the CT scanning device.

Example 15 can include the method of any of examples 1-14, further comprising: prompting a user to place a positioning sticker on one or more of the upper denture or the lower denture prior to scanning.

Example 16 can include a method of preparing a digital denture model, the method comprising: receiving scan data comprising: a bite scan comprising a scan of an upper denture positioned with a lower denture in a patient bite orientation, an upper denture scan, and a lower denture scan; forming a bite model, an upper denture model and a lower denture model from the received bite scan, upper denture scan, and lower denture scan; determining a relative position of the upper denture model and the lower denture model based on the bite scan; and revising the upper denture model and the lower denture model to produce a denture design.

Example 17 can include the method of example 16, wherein forming the bite model, the upper denture model, and the lower denture model comprises converting the bite scan, the upper denture scan, and the lower denture scan to mesh representations of denture surfaces.

Example 18 can include the method of any one of examples 16-17, wherein the bite scan, the upper denture scan, and the lower denture scan are received as DICOM files, the method further comprising converting the DICOM files to STL files prior to determining the relative position of the upper denture scan and lower denture scan based on the bite scan.

Example 19 can include the method of any of examples 16-18, further comprising: adjusting a threshold to select a surface of the bite model, the upper denture model, and the lower denture model.

Example 20 can include the method of any of examples 16-19, further comprising: detecting a position of the bite scan, the upper denture scan, and the lower denture scan within a scanning field; and orienting the bite scan, the upper denture scan, and the lower denture scan based on the detected position.

Example 21 can include the method of any one of examples 16-20, wherein detecting a position of the bite scan, the upper denture scan, and the lower denture scan comprises detecting a presence and position of an indicator in scan data.

Example 22 can include the method of any one of examples 16-21, wherein the indicator is a radio-opaque sticker positioned on one or more of the upper denture and the lower denture.

Example 23 can include the method of any one of examples 16-22, wherein the indicator is a radio-opaque sticker in the scanning field.

Example 24 can include the method of any of examples 16-23, further comprising: removing irregularities from one or more of the bite model, the upper denture model, and the lower denture model.

Example 25 can include the method of any one of examples 16-24, wherein the irregularities are voids within the bite model, the upper denture model, and the lower denture model, or voids on a surface of the bite model, the upper denture model, and the lower denture model.

Example 26 can include the method of any one of examples 16-25, wherein the irregularities are metallic deposits in the bite model, the upper denture model, and the lower denture model.

Example 27 can include the method of any one of examples 16-26, wherein the metallic deposits are indicated by a different density material.

Example 28 can include the method of any of examples 16-27, further comprising: receiving a color image representing one or more of the upper denture and the lower denture; extracting color and pattern information from the color image; and overlaying the extracted color and pattern information on the upper denture model and the lower denture model.

Example 29 can include the method of any of examples 16-28, wherein the received bite scan includes a bite registration material separating an upper denture arch from a lower denture arch, and wherein the bite registration material comprises a material having a different density than a material forming the upper denture arch and lower denture arch.

Example 30 can include the method of any one of examples 16-29, further comprising: adjusting a threshold to remove the bite registration material from the bite scan prior to determining a relative position of the upper denture model and the lower denture model based on the bite model.

Example 31 can include the method of any of examples 16-30, wherein the bite scan, the upper denture scan, and the lower denture scan are obtained by CT scanning device.

Example 32 can include the method of any of examples 16-31, wherein the bite scan, the upper denture scan, and the lower denture scan are obtained by CBCT scanning device.

Example 33 can include the method of any of examples 16-32, further comprising: detecting an identification of each of the bite scan, the upper denture scan, and the lower denture scan.

Example 34 can include the method of any one of examples 16-33, wherein the detecting an identification is based on a comparison of the scan data.

Example 35 can include the method of any of examples 16-34, further comprising transmitting for display an indication of scan quality.

Example 36 can include a method of preparing a digital denture model, the method comprising: receiving an upper denture scan and a lower denture scan; forming an upper denture model and a lower denture model from the received upper denture scan and the received lower denture scan; determining an initial bite position of the upper denture model and the lower denture model; and revising the upper denture model and the lower denture model to produce a denture design.

Example 37 can include the method of example 36, further comprising: presenting the initial bite position of the upper denture model and the lower denture model to a user.

Example 38 can include a method of preparing a digital denture model, the method comprising: receiving a bite scan comprising a scan of an upper denture and lower denture positioned together in a patient bite orientation; forming a bite model from the received bite scan; separating the bite model into an upper denture model and a lower denture model; and revising the upper denture model and the lower denture model to produce a denture design.

Example 39 can include the method of example 38, wherein the bite scan further comprises a bite registration material between the upper denture and the lower denture.

Example 40 can include the method of any one of examples 38-39, further comprising: identifying a presence of a bite registration material based on a density of materials in the bite scan; and adjusting a threshold to remove the bite registration material from the bite scan.

Example 41 can include a method of using a CT scanning device to obtain scanned denture data; the method comprising: positioning an upper denture and lower denture together in a patient bite orientation; positioning the upper and lower denture in the patient bite orientation in a scanning area of the CT scanning device; performing a CT scan with the CT scanning device; confirming an identification of the CT scan as the patient bite orientation in a user interface coupled to the CT scanning device; and receiving, in the user interface, a quality indication associated with the performed CT scan.

Example 42 can include the method of example 41, further comprising: positioning the upper denture in the scanning area of the CT scanning device; obtaining, using the CT scanning device, an upper denture scan; positioning the lower denture in the scanning area of the CT scanning device; obtaining, using the CT scanning device, a lower denture scan.

Example 43 can include the method of any one of examples 41-42, wherein positioning the upper denture and the lower denture together in a patient bite orientation in the scanning area of the CT scanning device comprises aligning a midline of the upper denture and the lower denture with a fiducial in the scanning area.

Example 44 can include the method of any one of examples 41-43, further comprising: positioning a bite registration material between the upper denture and the lower denture prior to positioning the upper denture and the lower denture together in the patient bite orientation.

Example 45 can include the method of any one of examples 41-44, further comprising: preparing the bite registration material by: positioning a bite registration material between the upper denture and the lower denture while the upper denture and the lower denture are in a patient's mouth; preparing a bite impression in the bite registration material while the upper denture and the lower denture are in a patient's mouth; and after preparing the bite impression, removing the upper denture, the lower denture, and the bite registration material from the patient's mouth.

Example 46 can include the method of any one of examples 41-45, wherein positioning the upper denture and the lower denture together in the patient bite orientation further comprises using the bite impression to align the upper denture and the lower denture in the patient bite orientation.

Example 47 can include the method of any one of examples 41-46, wherein the bite registration material is a radio-opaque material.

Example 48 can include the method of any one of examples 41-47, wherein the bite registration material is a radio-translucent material.

Example 49 can include the method of any one of examples 41-48, wherein the bite registration material has a known density different than a density of a material forming the upper denture and the lower denture.

Example 50 can include the method of any one of examples 41-49, further comprising: obtaining, using a color camera, a color image of the upper denture and the lower denture together in a patient bite orientation.

Example 51 can include the method of any one of examples 41-50, wherein the color image is obtained by a camera coupled to the CT scanning device.

Example 52 can include the method of any one of examples 41-51, wherein obtaining the color image is performed simultaneously with performing the CT scan of the upper and lower denture in the patient bite orientation.

Example 53 can include the method of any one of examples 41-52, wherein the CT scanning device is a cone beam CT scanning device.

Example 54 can include the method of any one of examples 41-53, further comprising: adjusting a field of view of the CT scanning device to include all features of the upper denture and the lower denture together in the patient bite orientation prior to obtaining the bite scan.

Example 55 can include the method of any one of examples 41-54, further comprising: re-adjusting the field of view of the CT scanning device after performing the bite scan in response to a notification that the field of view of the first scan does not include all features of the upper denture and the lower denture together in the patient bite orientation.

Example 56 can include the method of any one of examples 41-55, further comprising: placing a positioning fiducial on one or more of the upper denture or the lower denture prior to scanning.

Example 57 can include a method of generating a digital denture model, the method comprising: providing, on a user interface associated with a CT scanning device, a prompt to position an upper denture and a lower denture together in a patient bite orientation in a scanning area of the CT scanning device; performing a CT scan of the upper denture and the lower denture together in the patient bite orientation using the CT scanning device; receiving a bite scan based on the CT scan of the upper denture and the lower denture together in the patient bite orientation; and performing a quality analysis of the bite scan and providing a result of the quality analysis on a display.

Example 58 can include the method of example 57, further comprising: providing a prompt, on the user interface, to position an upper denture alone in the scanning area of the CT scanning device; performing a CT scan of the upper denture; providing a prompt, on the user interface, to position a lower denture alone in the scanning area of the CT scanning device.

Example 59 can include the method of any one of examples 57-58, further comprising: generating a model of a denture surface based on the bite scan, the upper denture scan, and the lower denture scan.

Example 60 can include the method of any one of examples 57-59, further comprising: orienting the upper denture scan relative to the lower denture scan based on the bite scan; and preparing the model of the denture surface based on the oriented upper denture and lower denture, where the upper denture and the lower denture are oriented according to an orientation in the bite scan.

Example 61 can include the method of any one of examples 57-60, further comprising: detecting, in the bite scan, a presence of a bite registration material positioned between the upper denture and the lower denture.

Example 62 can include the method of any one of examples 57-61, wherein detecting the presence of the bite registration material further comprises detecting a material having a different density than a density of one or more materials of the upper denture and the lower denture.

Example 63 can include the method of any one of examples 57-62, wherein the bite registration material is a radio-opaque material.

Example 64 can include the method of any one of examples 57-63, wherein the bite registration material is a radio-translucent material.

Example 65 can include the method of any one of examples 57-64, further comprising: adjusting a threshold of the model of the denture surface to remove the bite registration material.

Example 66 can include the method of any one of examples 57-65, further comprising: receiving a color image of the upper denture and the lower denture together in the patient bite orientation; extracting color and pattern information from the received color image; and correlating the color and pattern information with the model of the denture surface.

Example 67 can include the method of any one of examples 57-66, further comprising: generating a prompt to adjust a field of view of the CT scanning device based on a quality analysis of the bite scan, where a result of the quality analysis indicates that not all features of the bite scan are included in a current field of view.

Example 68 can include the method of any one of examples 57-67, further comprising: presenting, on the user interface, an image of the bite scan.

Example 69 can include the method of any one of examples 57-68, further comprising: presenting, on the user interface, an indication of scan quality with the image of the bite scan.

Example 70 can include the method of any one of examples 57-69, further comprising: prompting, on the user interface, a user to identify a scan as one of the bite scan, the upper denture scan, and the lower denture scan.

Example 71 can include the method of any one of examples 57-70, further comprising: presenting, on the user interface, a suggested identification of a scan as one of the bite scan, the upper denture scan, and the lower denture scan, wherein the suggested identification is based on an analysis of the scan resulting in a categorization of the scan.

Example 72 can include the method of any one of examples 57-71, further comprising: prompting a user, on the user interface, to position one or more of the upper denture and the lower denture relative to an indicator in the scanning area of the CT scanning device.

Example 73 can include the method of any one of examples 57-72, further comprising: prompting a user, on the user interface, to place a positioning sticker on one or more of the upper denture or the lower denture prior to scanning.

Example 74 can include a method of preparing a digital denture model, the method comprising: receiving scan data comprising: a bite scan comprising a scan of an upper denture positioned with a lower denture in a patient bite orientation, an upper denture scan, and a lower denture scan; forming a bite model, an upper denture model and a lower denture model from the received bite scan, upper denture scan, and lower denture scan; determining a relative position of the upper denture model and the lower denture model based on the bite scan; and generating a denture model based on the upper denture model, lower denture model, and determined relative position.

Example 75 can include the method of example 74, wherein forming the bite model, the upper denture model, and the lower denture model comprises converting the bite scan, the upper denture scan, and the lower denture scan to mesh representations of denture surfaces.

Example 76 can include the method of any one of examples 74-75, wherein the bite scan, the upper denture scan, and the lower denture scan are received as DICOM files, the method further comprising converting the DICOM files to one of an STL file, PLY file, OBJ file, AMF file, and 3MF file prior to determining the relative position of the upper denture scan and lower denture scan based on the bite scan.

Example 77 can include the method of any one of examples 74-76, further comprising: adjusting a threshold to select a surface of the bite model, the upper denture model, and the lower denture model.

Example 78 can include the method of any one of examples 74-77, further comprising: detecting a position of the bite scan, the upper denture scan, and the lower denture scan within a scanning field; and orienting the bite scan, the upper denture scan, and the lower denture scan based on the detected position.

Example 79 can include the method of any one of examples 74-78, wherein detecting a position of the bite scan, the upper denture scan, and the lower denture scan comprises detecting a presence and position of an indicator in scan data.

Example 80 can include the method of any one of examples 74-79, wherein the indicator is a radio-opaque sticker positioned on one or more of the upper denture and the lower denture.

Example 81 can include the method of any one of examples 74-80, wherein the indicator is a radio-opaque sticker in the scanning field.

Example 82 can include the method of any one of examples 74-81, further comprising: removing irregularities from one or more of the bite model, the upper denture model, and the lower denture model.

Example 83 can include the method of any one of examples 74-82, wherein the irregularities are voids within the bite model, the upper denture model, and the lower denture model, or voids on a surface of the bite model, the upper denture model, and the lower denture model.

Example 84 can include the method of any one of examples 74-83, wherein the irregularities are metallic deposits in the bite model, the upper denture model, and the lower denture model.

Example 85 can include the method of any one of examples 74-84, wherein the metallic deposits are indicated by a different density material.

Example 86 can include the method of any one of examples 74-85, further comprising: receiving a color image representing one or more of the upper denture and the lower denture; extracting color and pattern information from the color image; and correlating the extracted color and pattern information on the upper denture model and the lower denture model.

Example 87 can include the method of any one of examples 74-86, wherein the received bite scan includes a bite registration material separating an upper denture arch from a lower denture arch, and wherein the bite registration material comprises a material having a different density than a material forming the upper denture arch and lower denture arch.

Example 88 can include the method of any one of examples 74-87, further comprising: adjusting a threshold to remove the bite registration material from the bite scan prior to determining a relative position of the upper denture model and the lower denture model based on the bite model.

Example 89 can include the method of any one of examples 74-88, wherein the bite scan, the upper denture scan, and the lower denture scan are obtained by CT scanning device.

Example 90 can include the method of any one of examples 74-89, wherein the bite scan, the upper denture scan, and the lower denture scan are obtained by CBCT scanning device.

Example 91 can include the method of any one of examples 74-90, further comprising: detecting an identification of each of the bite scan, the upper denture scan, and the lower denture scan.

Example 92 can include the method of any one of examples 74-91, wherein the detecting an identification is based on a comparison of the scan data.

Example 93 can include the method of any one of examples 74-92, further comprising generating for display an indication of scan quality.

Example 94 can include a method of preparing a digital denture model, the method comprising: receiving an upper denture scan and a lower denture scan; forming an upper denture model and a lower denture model from the received upper denture scan and the received lower denture scan; determining an initial bite position of the upper denture model and the lower denture model; and generating a denture model based on the upper denture model, the lower denture model and the initial bite position.

Example 95 can include the method of example 94, further comprising: presenting the initial bite position of the upper denture model and the lower denture model in a user interface.

Example 96 can include a method of preparing a digital denture model, the method comprising: receiving a bite scan comprising a scan of an upper denture and lower denture positioned together in a patient bite orientation; forming a bite model from the received bite scan; separating the bite model into an upper denture model and a lower denture model; and generating a denture model based on the upper denture model, the lower denture model, and a positioning of the upper denture model and lower denture model based on the bite model orientation.

Example 97 can include the method of example 96, wherein the bite scan further comprises a bite registration material between the upper denture and the lower denture.

Example 98 can include the method of any one of examples 96-97, further comprising: identifying a presence of a bite registration material based on a density of materials in the bite scan; and adjusting a threshold to remove the bite registration material from the bite scan.

What is claimed is:

1. A method of using a CT scanning device to obtain scanned denture data, the method comprising:
positioning an upper denture and lower denture together in a patient bite orientation;
positioning the upper and lower denture in the patient bite orientation in a scanning area of the CT scanning device;
performing a CT scan with the CT scanning device;
confirming an identification of the CT scan as the patient bite orientation in a user interface coupled to the CT scanning device; and
receiving, in the user interface, a quality indication associated with the performed CT scan.

2. The method of claim 1, further comprising:
positioning the upper denture in the scanning area of the CT scanning device;
obtaining, using the CT scanning device, an upper denture scan;
positioning the lower denture in the scanning area of the CT scanning device; and
obtaining, using the CT scanning device, a lower denture scan.

3. The method of claim 1, wherein positioning the upper denture and the lower denture together in a patient bite orientation in the scanning area of the CT scanning device comprises aligning a midline of the upper denture and the lower denture with a fiducial in the scanning area.

4. The method of claim 1, further comprising:
positioning a bite registration material between the upper denture and the lower denture prior to positioning the upper denture and the lower denture together in the patient bite orientation; and
preparing the bite registration material by:
positioning a bite registration material between the upper denture and the lower denture while the upper denture and the lower denture are in a patient's mouth;
preparing a bite impression in the bite registration material while the upper denture and the lower denture are in a patient's mouth; and
after preparing the bite impression, removing the upper denture, the lower denture, and the bite registration material from the patient's mouth,
wherein positioning the upper denture and the lower denture together in the patient bite orientation further comprises using the bite impression to align the upper denture and the lower denture in the patient bite orientation.

5. The method of claim 4, wherein the bite registration material is at least one of (i) a radio-opaque material and (ii) a radio-translucent material.

6. The method of claim 4, wherein the bite registration material has a known density different than a density of a material forming the upper denture and the lower denture.

7. The method of claim 1, further comprising:
obtaining, using a color camera that is coupled to the CT scanning device, a color image of the upper denture and the lower denture together in a patient bite orientation, wherein obtaining the color image is performed simultaneously with performing the CT scan of the upper and lower denture in the patient bite orientation.

8. The method of claim 1, further comprising:
adjusting a field of view of the CT scanning device to include all features of the upper denture and the lower denture together in the patient bite orientation prior to obtaining the bite scan; and
re-adjusting the field of view of the CT scanning device after performing the bite scan in response to a notification that the field of view of the first scan does not include all features of the upper denture and the lower denture together in the patient bite orientation.

9. A method of generating a digital denture model, the method comprising:
providing, on a user interface associated with a CT scanning device, a prompt to position an upper denture and a lower denture together in a patient bite orientation in a scanning area of the CT scanning device;
performing a CT scan of the upper denture and the lower denture together in the patient bite orientation using the CT scanning device;
receiving a bite scan based on the CT scan of the upper denture and the lower denture together in the patient bite orientation; and
performing a quality analysis of the bite scan and providing a result of the quality analysis on a display.

10. The method of claim 9, further comprising:
providing a prompt, on the user interface, to position an upper denture alone in the scanning area of the CT scanning device;
performing a CT scan of the upper denture;
providing a prompt, on the user interface, to position a lower denture alone in the scanning area of the CT scanning device;
generating a model of a denture surface based on the bite scan, the upper denture scan, and the lower denture scan, wherein generating the model comprises:
orienting the upper denture scan relative to the lower denture scan based on the bite scan; and
preparing the model of the denture surface based on the oriented upper denture and lower denture, where the upper denture and the lower denture are oriented according to an orientation in the bite scan.

11. The method of claim 9, further comprising:
detecting, in the bite scan, a presence of a bite registration material positioned between the upper denture and the lower denture based on detecting a material having a different density than a density of one or more materials of the upper denture and the lower denture.

12. The method of claim 11, further comprising:
adjusting a threshold of the model of the denture surface to remove the bite registration material.

13. The method of any of claim 9, further comprising:
presenting, on the user interface, at least one of (i) an image of the bite scan and (ii) an indication of scan quality with the image of the bite scan; and
generating a prompt to adjust a field of view of the CT scanning device based on a quality analysis of the bite scan, where a result of the quality analysis indicates that not all features of the bite scan are included in a current field of view, wherein the result comprises the indication of the scan quality.

14. The method of claim 9, further comprising:
presenting, on the user interface, a suggested identification of a scan as one of the bite scan, the upper denture scan, and the lower denture scan, wherein the suggested identification is based on an analysis of the scan resulting in a categorization of the scan;
prompting a user, on the user interface, to position one or more of the upper denture and the lower denture relative to an indicator in the scanning area of the CT scanning device; and
prompting the user, on the user interface, to place a positioning sticker on one or more of the upper denture or the lower denture prior to scanning.

15. A method of preparing a digital denture model, the method comprising:
receiving scan data comprising:
a bite scan comprising a scan of an upper denture positioned with a lower denture in a patient bite orientation,
an upper denture scan, and
a lower denture scan;
forming a bite model, an upper denture model and a lower denture model from the received bite scan, upper denture scan, and lower denture scan, wherein forming the bite model, the upper denture model, and the lower denture model comprises converting the bite scan, the upper denture scan, and the lower denture scan to mesh representations of denture surfaces;
determining a relative position of the upper denture model and the lower denture model based on the bite scan; and
generating a denture model based on the upper denture model, lower denture model, and determined relative position.

16. The method of claim 15, wherein the bite scan, the upper denture scan, and the lower denture scan are received as DICOM files, the method further comprising converting the DICOM files to one of an STL file, PLY file, OBJ file, AMF file, and 3MF file prior to determining the relative position of the upper denture scan and lower denture scan based on the bite scan.

17. The method of claim 15, further comprising:
adjusting a threshold to select a surface of the bite model, the upper denture model, and the lower denture model.

18. The method of claim 15, further comprising:
detecting a position of the bite scan, the upper denture scan, and the lower denture scan within a scanning field based on detecting a presence and position of an indicator in scan data, wherein the indicator is a radio-opaque sticker (i) positioned on one or more of the upper denture and the lower denture or (i) in the scanning field; and
orienting the bite scan, the upper denture scan, and the lower denture scan based on the detected position.

19. The method of claim 15, further comprising:
removing irregularities from one or more of the bite model, the upper denture model, and the lower denture model, wherein the irregularities are at least one of (i) voids within the bite model, the upper denture model, and the lower denture model, (ii) voids on a surface of the bite model, the upper denture model, and the lower denture model, and (iii) metallic deposits in the bite model, the upper denture model, and the lower denture model, wherein the metallic deposits are indicated by a different density material.

20. The method of claim 15, further comprising:
receiving a color image representing one or more of the upper denture and the lower denture;
extracting color and pattern information from the color image; and
correlating the extracted color and pattern information on the upper denture model and the lower denture model.
